(19) United States
(12) Patent Application Publication
(10) Pub. No.: US 2013/0210715 A1
(43) Pub. Date: Aug. 15, 2013

(54) NUTRITIONAL COMPOSITIONS AND METHODS FOR WEANING FROM PARENTERAL NUTRITION TO ENTERAL NUTRITION

(71) Applicants: Norman Alan Greenberg, New Hope, MN (US); Claudia Roessle, Morges (CH)

(72) Inventors: Norman Alan Greenberg, New Hope, MN (US); Claudia Roessle, Morges (CH)

(73) Assignee: NESTEC S.A., Vevey (CH)

(21) Appl. No.: 13/643,350

(22) PCT Filed: Apr. 25, 2011
(86) PCT No.: PCT/US11/33729

§ 371 Date: Dec. 20, 2012

(30) Foreign Application Priority Data
Apr. 26, 2010 (US) 61327883
Mar. 1, 2011 (US) 61448171
Mar. 22, 2011 (US) 61466017

(51) Int. Cl. A61K 9/00; A23L 1/29

(52) U.S. Cl. CPC A61K 9/0029; A23L 1/296
USPC 514/5.6; 514/5.7

(57) ABSTRACT

Nutritional compositions and methods of using the nutritional compositions are provided. In a general embodiment, the present disclosure provides a nutritional composition including one or more proteins, one or more amino acids and one or more exogenous nucleotides. The nutritional compositions can be specifically used to assist a patient in weaning from partial or total parenteral nutrition to enteral nutrition with improved tolerance.

### BACKGROUND

The present disclosure generally relates to health and nutrition. More specifically, the present disclosure relates to nutritional compositions for weaning from total parenteral nutrition to enteral nutrition and methods of making and using the nutritional compositions.

There are many types of nutritional compositions currently on the market. Nutritional compositions can be targeted toward certain consumer types, for example, young, elderly, athletic, etc., based on the specific ingredients of the nutritional composition. Nutritional compositions can also be formulated based on the certain physiological conditions that the nutritional compositions are intended to treat or improve.

The use of partial parenteral nutrition or total parenteral nutrition occurs when a patient is unable to consume food orally and the gastrointestinal (“GI”) tract is not able to tolerate enteral tube feeding. This is usually due to hemodynamic instability. This refers to unstable blood pressure, especially dangerously low blood pressure, also called hypotension, which can involve a lack of blood flow to support the various organs in the body.

There are also certain medical conditions where the intestinal tract should not be used, which include, for example, healing of an anastomosis, or other GI surgeries, or severe inflammatory bowel disease such as acute flare-ups of Crohn's Disease. It is also necessary to use parenteral nutrition for patients with short bowel syndrome if extensive diarrhea has occurred so that the patient receives adequate nutrition and fluids.

During the time when the GI tract is not used, it atrophies in the same way that other unused body parts atrophy with disuse. This disuse causes the GI tract to potentially become a source of infection due to the reduction in GI tract integrity. To alleviate this risk and to provide better support to the immune components in the GI tract, it is accepted clinical practice to provide enteral feeding as soon as the patient is able to tolerate it.

Currently, there is no specific formula that is designed to wean patients from total parenteral nutrition to enteral nutrition. The present practice is to use a slow rate of feeding into the GI tract. Nevertheless, the patient can suffer from GI problems during the switch from total parenteral nutrition to enteral nutrition.

### SUMMARY

Nutritional compositions for weaning from parenteral nutrition to enteral nutrition and methods of making and using the nutritional compositions are provided. In a general embodiment, the present disclosure provides a nutritional composition including one or more proteins, one or more amino acids and one or more exogenous nucleotides. For example, the nutritional composition includes ingredients that support the repair, function, and reversion of atrophy of the small bowel in particular, as well as other ingredients that support the repair, function, and reversion of atrophy of the large bowel, which can assist the transition of a patient from partial parenteral nutrition or total parenteral nutrition to enteral nutrition. As a result, the transition can occur more efficiently (e.g., less diarrhea, better tolerance, and targeted enteral feeding goals can be achieved more rapidly).

In an embodiment, the protein is an intact protein, free amino acids, a whey protein hydrolysate, casein hydrolysate, milk protein hydrolysate, soy protein hydrolysate, pea protein hydrolysate or a combination thereof. The protein can range from about 35% to about 55% of the total energy of the nutritional composition. Offering a patient different types of proteins can optimize absorption.

In an embodiment, the amino acid is provided by a glutamine source, a threonine source, a serine source, a proline source, a cysteine source or a combination thereof. The glutamine source can be a glutamine dipeptide and/or glutamine enriched wheat protein. The amino acid can be in an amount from about 20 grams to about 40 grams in the nutritional composition.

In an embodiment, the exogenous nucleotide is in a monomeric form such as 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate or a combination thereof. The exogenous nucleotide can also be intact ribonucleic acid. The exogenous nucleotide can be in an amount of about 1 to about 4 grams in the nutritional composition.

In an embodiment, the nutritional composition includes one or more lipids. The lipid can be short chain triglycerides, medium chain triglycerides, long chain triglycerides, fish oil, vegetable oil or a combination thereof. In an embodiment, the lipids include tributyrin.

Experiments have shown that long-chain polyunsaturated fatty acids (“LC-PUFAs”) are absorbed more readily in the form of triglycerides (“TG”). Examples of LC-PUFAs include omega-3 fatty acids such as eicosapentaenoic acid (“EPA”), and docosahexaenoic acid (“DHA”), and omega-6 fatty acids such as gamma-linolenic acid (“GLA”), dihomo-gamma-linolenic acid (“DGLA”), and arachidonic acid (“ARA”).

Experiments have also shown that monoglycerides (“MG”) are readily absorbed even from compromised GI tracts. There are two types of MG, sn-2 and sn-1 (sn-3 and sn-1 are equivalent). Previous work had shown that the fatty acid in the sn-2 position is absorbed more quickly than the fatty acid in the sn-1 position when TG are administered. MG as both forms, sn-1 and sn-2 are both readily absorbed. The use of a MG will help to maximize lipid absorption which can help to improve the transition from parenteral to enteral support.

In an embodiment, the nutritional composition includes an ingredient such as synbiotics, phytonutrients or a combination thereof. Additional ingredients in the nutritional composition can include lipids, carbohydrates, antioxidants, vitamins, minerals, or a combination thereof.

In an embodiment, the nutritional composition includes one or more fibers. The fiber can be galacto-oligosaccharides, fructo-oligosaccharides, fuco-oligosaccharides, xylo-oligosaccharides, palatinose-oligosaccharide, soybean oligosaccharide, gentio-oligosaccharide, inulin, pectin, pectate, alginate, chondroitine, hyaluronic acids, heparine, heparane, sialoglycans, fucoidan, carrageenan, xanthan gum, cellulose, polydextrose, guar gum, partially hydrolyzed guar gum or a combination thereof.

In an embodiment, the nutritional composition includes one or more probiotics. The probiotic can be Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof. In another embodiment, the nutritional composition includes one or more non-replicating microorganisms.

In an embodiment, the nutritional composition is in an administrable form such as pharmaceutical formulations, nutritional formulations, dietary supplements, functional foods and beverage products.

In another embodiment, the present disclosure provides a method of making a nutritional composition. The method comprises combining one or more proteins, one or more amino acids and one or more exogenous nucleotides to form a nutritional composition.

In an alternative embodiment, the present disclosure provides a method of weaning a patient from parenteral nutrition administration to enteral nutrition administration. The method comprises administering to a patient having previously received nutrition parenterally a weaning nutritional composition including a protein, an amino acid and an exogenous nucleotide, and enterally administering to the patient an enteral nutritional composition.

In an embodiment, the method of weaning the patient comprises administering about 500 mL of the weaning nutritional composition during an initial 24 hour period before administering the enteral nutritional composition in greater volume. The method can further comprise administering about 750 mL of the weaning nutritional composition during a second subsequent 24 hour period before administering an enteral nutritional composition. The method can further comprise administering about 1000 mL of the weaning nutritional composition through a third subsequent 24 hour period before administering an enteral nutritional composition for additional days of feeding.

In yet another embodiment, the present disclosure provides a method of providing nutrition to a patient. The method comprises providing parenteral nutrition to a patient and administering to the patient having previously received nutrition parenterally a weaning nutritional composition comprising a protein, an amino acid and an exogenous nucleotide. In another embodiment, the methods include simultaneously administering to a patient receiving nutrition parenterally, a weaning nutritional composition comprising a protein, an amino acid and an exogenous nucleotide. The parenteral nutrition can be total parenteral nutrition (e.g., no food is given by any other routes other than intravenously).

In an embodiment of any of the methods, the protein is an intact protein, free amino acids, a whey protein hydrolysate, casein hydrolysate, milk protein hydrolysate, soy protein hydrolysate, pea protein hydrolysate or a combination thereof. The protein can range from about 35% to about 55% of the total energy of the nutritional composition.

In an embodiment of any of the methods, the amino acid is from a glutamine source, a threonine source, a cysteine source, a serine source, a proline source, or a combination thereof. The glutamine source can be a glutamine dipeptide, a glutamine enriched wheat protein or a combination thereof. The amino acid can be in an amount from about 20 grams to about 40 grams in the nutritional composition.

In an embodiment of any of the methods, the exogenous nucleotide is in a monomeric form such as 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate or a combination thereof. The exogenous nucleotide can also be intact ribonucleic acid. The exogenous nucleotide can be in an amount of about 1 to about 4 grams in the nutritional composition.

In an embodiment of any of the methods, the nutritional composition includes one or more lipids. The lipid can be short chain triglycerides, medium chain triglycerides, long chain triglycerides, fish oil, vegetable oil or a combination thereof. In an embodiment, the lipids include tributyrin.

In an embodiment of any of the methods, the nutritional composition includes an ingredient such as synbiotics, phytonutrients or a combination thereof. Additional ingredients in the nutritional composition can include lipids, carbohydrates, antioxidants, vitamins, minerals or a combination thereof.

In an embodiment of any of the methods, the nutritional composition includes one or more fibers. The fiber can be galacto-oligosaccharides, fructo-oligosaccharides, fuco-oligosaccharides, xylo-oligosaccharides, palatinose-oligosaccharide, soybean oligosaccharide, gentio-oligosaccharide, inulin, pectin, pectate, alginate, chondroitine, hyaluronic acids, heparine, heparane, sialoglycans, fucoidan, carrageenan, xanthan gum, cellulose, polydextrose, guar gum, partially hydrolyzed guar gum or a combination thereof.

In an embodiment of any of the methods, the nutritional composition includes one or more probiotics. The probiotic can be Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof. The probiotics may include non-replicating microorganisms.

In an embodiment of any of the methods, the nutritional composition is in an administrable form such as pharmaceutical formulations, nutritional formulations, dietary supplements, functional foods and beverage products.

An advantage of the present disclosure is to provide an improved nutritional composition having proteins (e.g., intact and/or hydrolyzed), amino acids and exogenous nucleotides.

Another advantage of the present disclosure is to provide a method of making an improved nutritional composition for weaning from total parenteral nutrition.

Yet another advantage of the present disclosure is to provide a nutritional composition that assists a patient in weaning from total parenteral nutrition to enteral nutrition with improved tolerance.

Still another advantage of the present disclosure is to provide a quicker transition to enteral nutrition for patients receiving total parenteral nutrition.

Another advantage of the present disclosure is to provide a reduced recovery time for a patient's normal digestive function after changing from total parenteral nutrition to enteral nutrition.

Additional features and advantages are described herein, and will be apparent from the following Detailed Description.

### DETAILED DESCRIPTION

As used herein, “about” is understood to refer to numbers in a range of numerals. Moreover, all numerical ranges herein should be understood to include all integer, whole or fractions, within the range.

As used herein the term “amino acid” is understood to include one or more amino acids. The amino acid can be, for example, alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

As used herein, “animal” includes, but is not limited to, mammals, which include but is not limited to, rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals that are capable of the effect exhibited or intended to be exhibited by the context of the passage.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione Goji (wolfberry), hesperidin, lactowolfberry, lignan, lutein, lycopene, polyphenols, selenium, vitamin A, vitamin B₁, vitamin B₆, vitamin B₁₂, vitamin C, vitamin D, vitamin E, zeaxanthin, or combinations thereof.

As used herein, “complete nutrition” includes nutritional products and compositions that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

While the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, non-limiting examples of fatty acid components of fish oils include docosahexaenoic acid (“DHA”) and eicosapentaenoic acid (“EPA”). Additional sources of DHA and EPA include plant sources of omega 3, flaxseed, walnut, algae, and krill.

As used herein, “food grade micro-organisms” means micro-organisms that are used and generally regarded as safe for use in food.

As used herein, “incomplete nutrition” includes nutritional products or compositions that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

As used herein, “long term administrations” are preferably continuous administrations for more than 6 weeks. Alternatively, “short term administrations,” as used herein, are continuous administrations for less than 6 weeks.

As used herein, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

The term “microorganism” is meant to include the bacterium, yeast and/or fungi, a cell growth medium with the microorganism, or a cell growth medium in which microorganism was cultivated.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, a “non-replicating” microorganism means that no viable cells and/or colony forming units can be detected by classical plating methods. Such classical plating methods are summarized in the microbiology book: James Monroe Jay, et al., Modern food microbiology, 7th edition, Springer Science, New York, N. Y. p. 790 (2005). Typically, the absence of viable cells can be shown as follows: no visible colony on agar plates or no increasing turbidity in liquid growth medium after inoculation with different concentrations of bacterial preparations (non replicating' samples) and incubation under appropriate conditions (aerobic and/or anaerobic atmosphere for at least 24 h). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

As used herein, a “nucleotide” is understood to be a subunit of deoxyribonucleic acid (“DNA”), ribonucleic acid (“RNA”), polymeric RNA, polymeric DNA, or combinations thereof. It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5′-Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. There can be multiple sources of the polymeric form such as, for example, yeast RNA.

“Nutritional products,” or “nutritional compositions,” as used herein, are understood to include any number of optional additional ingredients, including conventional food additives, for example one or more acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount. The nutritional products or compositions may be a source of complete nutrition or may be a source of incomplete nutrition.

As used herein the term “patient” is understood to include an animal, especially a mammal, and more especially a human that is receiving or intended to receive treatment, as it is herein defined.

As used herein, “phytochemicals” or “phytonutrients” are non-nutritive compounds that are found in many foods. Phytochemicals are functional foods that have health benefits beyond basic nutrition, are health promoting compounds that come from plant sources, and may be natural or purified. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3 -gallate, theaflavin-3′-gallate, theaflavin-3,3′-digallate, thearubigins), anthocyanins(flavonals) and anthocyanidins (such as, for example, pelargonidin, peonidin, cyanidin, delphinidin, malvidin, petunidin), isoflavones(phytoestrogens) (such as, for example, daidzein(formononetin), genistein(biochanin A), glycitein), dihydroflavonols, chalcones, coumestans(phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans(phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes(isoprenoids) which include carotenoids(tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and omega-3, 6, and 9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) combinations thereof.

As used in this disclosure and the appended claims, the singular forms “a,” “an” and “the” include plural referents unless the context clearly dictates otherwise. Thus, for example, reference to “a polypeptide” includes a mixture of two or more polypeptides, and the like.

As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn R. Gibson and Marcel B. Roberfroid, Dietary Modulation of the Human Colonic Microbiota: Introducing the Concept of Prebiotics, J. Nutr. 1995 125: 1401-1412. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

As used herein, probiotic micro-organisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al., Probiotics: how should they be defined?, Trends Food Sci. Technol. 1999:10, 107-10. In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of proteins include dairy based proteins, plant based proteins, animal based proteins and artificial proteins. Dairy based proteins include, for example, intact proteins, amino acids, casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, green pea powder, green bean powder, and any proteins derived from beans, lentils, and pulses. Animal based proteins may include, for example, beef, poultry, fish, lamb, seafood, or combinations thereof.

All dosage ranges contained within this application are intended to include all numbers, whole or fractions, contained within said range.

As used herein, a “synbiotic” is a supplement that contains both a prebiotic and a probiotic that work together to improve the microflora of the intestine.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

As used herein, a “tube feed” is a complete or incomplete nutritional product or composition that is administered to an animal's gastrointestinal system, other than through oral administration, including but not limited to a nasogastric tube, orogastric tube, gastric tube, jejunostomy tube (“J-tube”), percutaneous endoscopic gastrostomy (“PEG”), port, such as a chest wall port that provides access to the stomach, jejunum and other suitable access ports.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, analogs.

Nutritional compositions for weaning from total parenteral nutrition to enteral nutrition and methods of making and using the nutritional compositions are provided. The nutritional composition in embodiments of the present disclosure can support the GI tract during the transition from total parenteral nutrition to enteral nutrition so that there is a more effective weaning from total parenteral nutrition. This can be achieved by including several GI trophic ingredients that enable the small and large bowels to recover from the atrophy that occurred during total parenteral nutrition and resume functioning more quickly.

By administering the nutritional compositions in embodiments of the present disclosure, the rate of enteral feeding can be increased more quickly and the total parenteral nutrition discontinued sooner. This can allow for faster recovery of a patient's normal digestive function and a quicker hospital discharge for patients who were receiving total parenteral nutrition.

In a general embodiment, the nutritional composition includes one or more proteins, one or more amino acids and one or more exogenous nucleotides. The combination of specific proteins, amino acids and exogenous nucleotides can assist a patient (e.g., child, teenage, adult, elderly, animal) in weaning from total parenteral nutrition to enteral nutrition with improved tolerance.

In an embodiment, the nutritional composition has a low to moderate caloric density (e.g., 0.5-1.2 Kcal/mL) and provides a low to moderate feeding from about 0.5, 0.6, 0.7, 0.8, 0.9, 1.0, 1.1, or 1.2 Kcal/mL. It should be appreciated that any two amounts of the caloric density recited herein can further represent end points in a preferred range of the caloric densities of the nutritional composition. For example, the amounts of 0.7 and 0.9 Kcal/mL can represent the individual amounts of the caloric densities as well as a preferred range of the caloric densities between about 0.6 and 0.9 Kcal/mL.

The feeding volume may range depending on the size and weight of the patient or the patient's specific nutritional needs. For example, the feeding volumes may be anywhere between 100 ml to 1500 ml. The feeding volumes may be 100, 200, 300, 400, 500, 600, 700, 800, 900, 1000, 1100, 1200, 1300, 1400 and 1500. It should be appreciated that any two amounts of the feeding volumes recited herein can further represent end points in a preferred range of the feeding volumes of the nutritional composition. For example, the amounts of 600 ml and 1100 ml can represent the individual amounts of the feeding volumes as well as a preferred range of the feeding amounts between about 500 ml and about 1000 ml.

The macronutrient profile of the nutritional composition in an embodiment can be as follows: proteins—from about 35% to about 55% of the total energy; lipids—from about 20% to about 30% of the total energy; and carbohydrates—from about 15% or 20% to about 45% of the total energy of the nutritional composition.

The protein can mainly be provided as a hydrolysate. The protein can be a whey protein hydrolysate, casein hydrolysate, milk protein hydrolysate, soy protein hydrolysate, pea protein hydrolysate or a combination thereof. Whey protein, a preferred protein, is intrinsically rich in threonine and cysteine. However, additional amounts of threonine and/or soluble cysteine precursors (e.g., acetylcysteine, cystathionine) can be added as the amino acid as well. The protein may also be an intact protein, or free amino acids such as, but not limited to cysteine, or a source thereof. Cysteine sources may include, but are not limited to cystein-rich peptides, acetylcysteine, cystathionine, or combinations thereof.

The hydrolysates can have a low to high degree of hydrolysis. The hydrolysates contain a range of peptide sizes to act as trophic agents for the small bowel and allow the use of all intestinal transporter systems for peptides and amino acids thus facilitating optimum absorption of protein.

In an embodiment, the protein ranges from about 35% to about 55% of the total energy of the nutritional composition. More specifically, the amount of the protein can be about 35%, 40%, 45%, 50%, 55% and the like of the total energy. It should be appreciated that any two amounts of the protein recited herein can further represent end points in a preferred range of the protein in the nutritional composition. For example, the amounts of 40% and 50% by total energy can represent the individual amounts of the protein as well as a preferred range of the protein between about 40% and about 50% of the total energy.

The amino acid can be provided by a glutamine source, a threonine source, a cysteine source, a serine source, a proline source, or a combination thereof. The glutamine source can be a glutamine dipeptide and/or glutamine enriched wheat protein. Glutamine dipeptide can be included due to the use of glutamine by enterocytes as an energy source. Glutamine dipeptide offers the possibility of including glutamine in a liquid product. Threonine, serine and proline are important amino acids for the production of mucin. Mucin coats the GI tract and helps to protect it from attachment of pathogens. Cysteine is a major precursor of glutathione, which is key for the antioxidant defenses of the body that are needed in case of reperfusion of the intestine.

The amino acid can be in the nutritional composition in an amount ranging from about 20 grams to about 50 grams. More specifically, the amino acid can be in the nutritional composition in an amount of about 20 grams, 25 grams, 30 grams 35 grams, 40 grams, 45 grams, 50 grams and the like. It should be appreciated that any two amounts of the amino acid recited herein can further represent end points in a preferred range of the amino acid. For example, the amounts of 30 grams and 45 grams can represent the individual amounts of the amino acid in the nutritional composition as well as a preferred range of the amino acid between about 35 grams and about 40 grams in the nutritional composition.

Nucleotides are low molecular weight biological molecules key to biochemical processes. Sources include de novo synthesis, recovery via salvage mechanisms, and dietary intakes. While endogenous production serves as the main nucleotide source, there is evidence to suggest that exogenous sources are important in rapidly proliferating cells in the immune and gastrointestinal systems (e.g., epithelial and immune cells), where they may become conditionally essential. Exogenous nucleotides may support optimal growth and function of metabolically active cells in times of cellular insult and their supplementation may improve clinical outcomes in the critically ill and immune suppressed patient. In addition, non-protein nitrogen can come from nucleotides provided, for example, as yeast RNA.

The exogenous nucleotides can be in the form of monomers and polymers as part of the nutritional compositions. A nucleotide is a subunit of deoxyribonucleic acid (“DNA”) or ribonucleic acid (“RNA”). It is an organic compound made up of a nitrogenous base, a phosphate molecule, and a sugar molecule (deoxyribose in DNA and ribose in RNA). Individual nucleotide monomers (single units) are linked together to form polymers, or long chains. The exogenous nucleotides in embodiments of the present disclosure are specifically provided by dietary supplementation.

The exogenous nucleotides can be in a monomeric form such as, for example, 5′ Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”) or a combination thereof. The exogenous nucleotides can also be in a polymeric form such as, for example, an intact RNA. There can be multiple sources of the polymeric form such as, for example, yeast RNA.

The exogenous nucleotides can be in the nutritional composition an amount of about 1 to about 4 grams. More specifically, the exogenous nucleotides can be in the nutritional composition in an amount of about 1 gram, 1.2 grams, 1.4 grams, 1.6 grams, 1.8 grams, 2 grams, 2.2 grams, 2.4 grams, 2.6 grams, 2.8 grams, 3 grams, 3.2 grams, 3.4 grams, 3.6 grams, 3.8 grams, 4 grams and the like. It should be appreciated that any two amounts of the exogenous nucleotides recited herein can further represent end points in a preferred range of the exogenous nucleotides. For example, the amounts of 1.8 grams and 3.4 grams can represent the individual amounts of the exogenous nucleotides in the nutritional composition as well as a preferred range of the exogenous nucleotides between about 1.4 grams and about 3 grams in the nutritional composition.

Additional ingredients may be used with the proteins, amino acids and exogenous nucleotides to assist in the weaning process. In an embodiment, the nutritional composition includes one or more lipids. The lipid can be short chain triglycerides, medium chain triglycerides (“MCTs”), long chain triglycerides (“LCTs”), fish oil, vegetable oil or a combination thereof. The fish oils can include docosahexaenoic acid (“DHA”) and eicosapentaenoic acid (“EPA”). The vegetable oils can be, for example, canola oil, sunflower oil, safflower oil, corn oil, coconut oil, palm oil and soybean oil. In an embodiment, the lipids include tributyrin.

The lipid component can contain a high proportion of MCTs that are easy to absorb. As a total parenteral nutrition weaning composition, the content of essential fatty acids does not need to meet the typical daily long term needs of a human. Fish oil at a level to provide about 2 grams to about 3 grams of EPA and DHA serve to help control the inflammation associated with the ischemia/reperfusion associated with re-feeding of the GI tract. The overall n6:n3 ratio can be no higher than 5:1.

In an embodiment, the nutritional composition includes one or more fibers. The fiber can be galacto-oligosaccharides, fructo-oligosaccharides, fuco-oligosaccharides, xylo-oligosaccharides, palatinose-oligosaccharide, soybean oligosaccharide, gentio-oligosaccharide, inulin, pectin, pectate, alginate, chondroitine, hyaluronic acids, heparine, heparane, sialoglycans, fucoidan, carrageenan, xanthan gum, cellulose, polydextrose, partially hydrolyzed guar gum, guar gum or a combination thereof. Partially hydrolyzed guar gum, for example, has been proven to reduce the incidence of diarrhea in critical care patients. Fiber can be added as an optional ingredient as a means to improve feeding tolerance. If fiber is used, the fiber should be in an amount that prevents or minimizes intestinal discomfort.

In an embodiment, the nutritional composition includes one or more probiotics. The probiotic can be Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof. The probiotics may also include non-replicating microorganisms.

The nutritional composition can further include ingredients such as synbiotics, phytonutrients or a combination thereof. As used herein, a synbiotic is a supplement that contains both a prebiotic and a probiotic that work together to improve the microflora of the intestine. Non-limiting examples of phytonutrients include quercetin, curcumin, limonin, carotenoids, lutein, lycopene, etc.

Additional ingredients in the nutritional composition can include lipids, carbohydrates, antioxidants, vitamins, minerals or a combination thereof. Carbohydrates include various monosaccharides, disaccharides, oligosaccharides, and polysaccharides. Suitable carbohydrates include, for example, fast digesting maltodextrin, starch, corn syrup, glucose, sucrose, lactose, etc.

Antioxidants are molecules capable of slowing or preventing the oxidation of other molecules. Non-limiting examples of antioxidants include vitamin A, carotenoids, vitamin C, vitamin E, selenium, flavonoids, polyphenols, lycopene, lutein, lignan, coenzyme Q10 (“CoQ10”) and glutathione.

Non-limiting examples of vitamins may include Vitamins A, B-complex (such as B-1, B-2, B-6 and B-12), C, D, E and K, niacin and acid vitamins such as pantothenic acid and folic acid and biotin. Non-limiting examples of minerals may include calcium, iron, zinc, magnesium, iodine, copper, phosphorus, manganese, potassium, chromium, molybdenum, selenium, nickel, tin, silicon, vanadium and boron.

Vitamin C can be preferably used in the nutritional compositions for collagen synthesis, stimulation of interferon, maintenance of redox integrity of the cell and intracellular matrix components. Vitamin E can be preferably used in the nutritional compositions as a lipid soluble antioxidant that stabilizes cell membranes and prevents oxidative damage to immune cells. Selenium can be preferably used in the nutritional compositions as a component of selenoproteins to augment cellular immune response through production of interferon an earlier peak of T cell proliferation and increased T helper cells.

With the occurrence of ischemia/reperfusion, there is oxidative stress that could be met by a high level of antioxidant nutrients in embodiments of the nutritional composition. Vitamins C and E can be provided at two to ten times the accepted daily need of a healthy adult. In an embodiment, vitamin C is provided at four times the accepted daily need of a healthy adult, and vitamin E is provided at eight times the accepted daily need of a healthy adult. Selenium can also be provided at two or more times the daily requirement of a healthy adult. In an embodiment, selenium is provided at three times the daily requirement of a healthy adult.

Other optional ingredients (e.g., colors, flavors, spices, herbs, etc.) can be added to make the nutritional composition sufficiently palatable. The optional ingredients can be added in any suitable amount.

In an alternative embodiment, the present disclosure provides a method of weaning a patient from parenteral nutrition administration to enteral nutrition administration. The method comprises administering to a patient having previously received nutrition parenterally a weaning nutritional composition including a protein, an amino acid and an exogenous nucleotide, and enterally administering to the patient an enteral nutritional composition. The parenteral nutrition administration can be total parenteral nutrition administration (e.g., no food is given by any other routes other than intravenously). In yet another embodiment, methods of weaning a patient from parenteral administration to enteral nutrition administration. The method comprises administering to a patient undergoing a parenteral diet regimen a weaning nutritional compsoition including a protein, an amino acid and an exogenous nucleotide.

In an embodiment, the method of weaning the patient comprises administering about 500 mL of the weaning nutritional composition during an initial 24 hour period before enterally administering longer term a enteral nutritional composition. The method can further comprise administering about 750 mL of the weaning nutritional composition during a second subsequent 24 hour period before enterally administering longer term a enteral nutritional composition. The method can further comprise administering about 1000 mL of the weaning nutritional composition through a third subsequent 24 hour period before enterally administering longer term a enteral nutritional composition.

In yet another embodiment, the present disclosure provides a method of providing nutrition to a patient. The method comprises providing parenteral nutrition to a patient and administering to the patient having previously received nutrition parenterally a weaning nutritional composition comprising a protein, an amino acid and an endogenous nucleotide. The parenteral nutrition can be total parenteral nutrition.

Using any of the nutritional compositions previously described in embodiments of the present methods, the rate of enteral feeding should be able to be increased more quickly in a patient and the parenteral nutrition or total parenteral nutrition discontinued sooner. This is expected to allow for faster recovery of normal function and hospital discharge of the patient.

### EXAMPLES

By way of example and not limitation, the following examples are illustrative of various embodiments of the present disclosure. The formulations below are provided for exemplification only, and they can be modified by the skilled artisan to the necessary extent, depending on the special features that are desired. All percentages herein are based on the total weight of the composition unless noted otherwise.

### Example 1

Nutritional composition formulation #1

| | |
|---|---|
| Volume | 1,000 mL |
| Caloric density | 0.7 kcal/mL |
| Calories | 700 |
| Protein, including AA* | 50% of energy (includes dipeptide), whey |
| | hydrolysate |
| Glutamine (as Ala-Gln) | 20 g/L |
| Threonine | 3.0 g/L |
| Nucleotides | 1.5 g/L |
| Carbohydrates | 30% of energy |
| Lipids | 20% of energy |
| N6:N3 | maximum of 5:1 |
| MCT:LCT | 40:60 |
| Vitamins & minerals | Meet daily recommended intake (“DRI”) and |
| | safe and adequate levels in 1,000 mL (2 times |
| | the DRI for vitamins C and E and selenium) |
| Fiber | Without soluble or insoluble fiber that may |
| | cause bloating, etc. |

*AA = amino acids

### Example 2

Nutritional composition formulation #2

| | |
|---|---|
| Volume | 1,000 mL |
| Caloric density | 0.7 kcal/mL |
| Calories | 700 |
| Protein, including AA* | 50% of energy, whey hydrolysate |
| Glutamine (as Ala-Gln) | 20 g/L |
| Threonine | 2.0 g/L |
| Nucleotides | 1.5 g/L |
| Carbohydrates | 30% of energy |
| Lipids | 20% of energy |
| N6:N3 | maximum of 5:1 |
| MCT:LCT | 40:60 |
| Tributyrin | 4.0 g/L |
| Vitamins & minerals | food for Special Medical Purposes |
| Fiber | fiber free |

*AA = amino acids

### Example 3

Nutritional composition formulation #3

| | |
|---|---|
| Volume | 1,000 mL |
| Caloric density | 0.7 kcal/mL |
| Calories | 700 |
| Protein, including AA* | 55% of energy, whey hydrolysate |
| Glutamine (as Ala-Gln) | 20 g/L |
| Threonine | 2.0 g/L |
| Nucleotides | 1.5 g/L |
| Carbohydrates | 20% of energy |
| Lipids | 25% of energy |
| N6:N3 | maximum of 5:1 |
| MCT:LCT | 40:60 |
| Tributyrin | 10.0 g/L |
| Vitamins & minerals | food for special medical purposes |
| Fiber | fiber free |

*AA = amino acids

### Example 4

Nutritional composition formulation #4

| | |
|---|---|
| Volume | 1,000 mL |
| Caloric density | 0.7 kcal/mL |
| Calories | 700 |
| Protein, including AA* | 50% of energy, casein hydrolysate |
| Glutamine (as Ala-Gln) | 20 g/L |
| Threonine | 2.0 g/L |
| Cysteine | 2.0 g/L |
| Nucleotides | 1.5 g/L |
| Carbohydrates | 25% of energy |
| Lipids | 25% of energy |
| N6:N3 | maximum of 5:1 |
| MCT:LCT | 40:60 |
| Vitamins & minerals | food for special medical purposes except for |
| Vitamin C | 380 mg |
| Vitamin E | 120 mg |
| Selenium | 200 mcg |
| Fiber | fiber free |

*AA = amino acids

### Example 5

Nutritional composition formulation #5

| | |
|---|---|
| Volume | 1,000 mL |
| Caloric density | 0.8 kcal/mL |
| Calories | 800 |
| Protein, including AA* | 50% of energy, casein |
| Glutamine (as Ala-Gln) | 20 g/L |
| Threonine | 2.0 g/L |
| Nucleotides | 1.5 g/L |
| Carbohydrates | 30% of energy |
| Lipids | 20% of energy |
| N6:N3 | maximum of 5:1 |
| MCT:LCT | 40:60 |
| Vitamins & minerals | food for special medical purposes except for |
| Vitamin C | 380 mg |
| Vitamin E | 120 mg |
| Selenium | 200 mcg |
| Fiber | fiber free |

*AA = amino acids

### Examples 6 - 9

Examples 6 to 9 include the formulations of Examples 2 to 5, respectively, but wherein the protein source (e.g., whey hydrolysate, casein hydrolysate or casein) includes dipeptides.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional composition comprising a protein, an amino acid and an exogenous nucleotide:
the protein comprising from about 35% to about 55% of total energy of the nutritional composition and is selected from the group consisting of an intact protein, free amino acids, a whey protein hydrolysate, casein hydrolysate, milk protein hydrolysate, soy protein hydrolysate, pea protein hydrolysate, and combinations thereof;
the amino acid comprises from about 20 grams to about 40 grams and is selected from the group consisting of a glutamine source, a threonine source, a cysteine source, a serine source, a proline source, and combinations thereof; and
the exogenous nucleotide comprises about 1 to about 4 grams.

2. The nutritional composition according to claim 1, wherein the glutamine source is selected from the group consisting of a glutamine dipeptide, a glutamine enriched wheat protein, and combinations thereof.

3. The nutritional composition according to claim 1, wherein the exogenous nucleotide is in a monomeric form selected from the group consisting of 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate, and combinations thereof.

4. The nutritional composition according to claim 1, wherein the exogenous nucleotide is intact ribonucleic acid.

5. The nutritional composition according to claim 1, comprising at least one component selected from the group consisting of: lipids, carbohydrates, antioxidants, vitamins, minerals, fiber, synbiotics, probiotic, phytonutrients, and combinations thereof.

6. The nutritional composition according to claim 5, wherein the lipid is selected from the group consisting of short chain triglycerides, medium chain triglycerides, long chain triglycerides, fish oil, vegetable oil, and combinations thereof.

7. The nutritional composition according to claim 5, wherein the fiber is selected from the group consisting of galacto-oligosaccharides, fructo-oligosaccharides, fuco-oligosaccharides, xylo-oligosaccharides, palatinose-oligosaccharide, soybean oligosaccharide, gentio-oligosaccharide, inulin, pectin, pectate, alginate, chondroitine, hyaluronic acids, heparine, heparane, sialoglycans, fucoidan, carrageenan, xanthan gum, cellulose, polydextrose, partially hydrolyzed guar gum, guar gum, and combinations thereof.

8. The nutritional composition according to claim 5, wherein the probiotic is selected from the group consisting of Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus, and combinations thereof.

9. A method of making a nutritional composition, the method comprising: combining protein, amino acid and endogenous nucleotide in a ratio so that the protein comprises from about 35% to about 55% of total energy of the nutritional composition and is selected from the group consisting of an intact protein, free amino acids, a whey protein hydrolysate, casein hydrolysate, milk protein hydrolysate, soy protein hydrolysate, pea protein hydrolysate, and combinations thereof, the amino acid comprises from about 20 grams to about 40 grams and is selected from the group consisting of a glutamine source, a threonine source, a cysteine source, a serine source, a proline source, and combinations thereof, and the exogenous nucleotide comprises about 1 to about 4 grams to form a nutritional composition.

10. The method as claimed in claim 9, comprising combining at least one component selected from the group consisting of: lipids, carbohydrates, antioxidants, vitamins, minerals, fiber, synbiotics, probiotic, phytonutrients, and combinations thereof.

11. The method according to claim 9, wherein the nutritional composition is in an administerable form selected from the group consisting of pharmaceutical formulations, nutritional formulations, dietary supplements, functional foods, beverage products, and combinations thereof.

12. A method of providing nutrition to a patient, the method comprising:
providing parental nutrition to a patient; and
administering to the patient having previously received nutrition parenterally a weaning nutritional composition comprising a protein, an amino acid and an exogenous nucleotide, the protein comprising from about 35% to about 55% of total energy of the nutritional composition and is selected from the group consisting of an intact protein, free amino acids, a whey protein hydrolysate, casein hydrolysate, milk protein hydrolysate, soy protein hydrolysate, pea protein hydrolysate, and combinations thereof, the amino acid comprises from about 20 grams to about 40 grams and is selected from the group consisting of a glutamine source, a threonine source, a cysteine source, a serine source, a proline source, and combinations thereof, and the exogenous nucleotide comprises about 1 to about 4 grams.

13. The method according to claim 12, wherein the glutamine source is selected from the group consisting of a glutamine dipeptide, a glutamine enriched wheat protein, and combinations thereof.

14. The method according to claim 12, wherein the exogenous nucleotide is in a monomeric form selected from the group consisting of 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate, and combinations thereof.

15. The method according to claim 12, wherein the exogenous nucleotide is intact ribonucleic acid.

16. The method according to claim 12, wherein the weaning nutritional composition further comprising at least one component selected from the group consisting of lipids, carbohydrates, antioxidants, vitamins, minerals, fiber, synbiotics, probiotic, phytonutrients, and combinations thereof.

17. The method according to claim 16, wherein the lipid is selected from the group consisting of short chain triglycerides, medium chain triglycerides, long chain triglycerides, fish oil, vegetable oil, and combinations thereof.

18. The method according to claim 16, wherein the fiber is selected from the group consisting of galacto-oligosaccharides, fructo-oligosaccharides, fuco-oligosaccharides, xylo-oligosaccharides, palatinose-oligosaccharide, soybean oligosaccharide, gentio-oligosaccharide, inulin, pectin, pectate, alginate, chondroitine, hyaluronic acids, heparine, heparane, sialoglycans, fucoidan, carrageenan, xanthan gum, cellulose, polydextrose, partially hydrolyzed guar gum, guar gum, and combinations thereof.

19. The method according to claim 16, wherein the probiotic is selected from the group consisting of Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus, and combinations thereof.

20. The method according to claim 12, comprising administering about 500 mL to about 1000 ml of the weaning nutritional composition during an initial 24 hour period before enterally administering longer term an enteral nutritional composition.

21. The method according to claim 12 wherein the patient is weaned from total parenteral nutrition administration to total enteral nutrition administration.

22. The method according to claim 12, wherein the nutritional composition is in an administerable form selected from the group consisting of pharmaceutical formulations, nutritional formulations, dietary supplements, functional foods, beverage products, and combinations thereof.
